(19) United States
(12) Patent Application Publication
(10) Pub. No.: US 2022/0177953 A1
(43) Pub. Date: Jun. 9, 2022

(54) DETECTION OF DOUBLE STRANDED NUCLEIC ACIDS IN SITU AND METHODS RELATED THERETO

(71) Applicant: ADVANCED CELL DIAGNOSTICS, INC., Newark, CA (US)

(72) Inventors: Xiao-Jun MA, Newark, CA (US); Farzaneh TONDNEVIS, Newark, CA (US); Courtney TODOROV, Newark, CA (US); Bingqing ZHANG, Newark, CA (US)

(73) Assignee: ADVANCED CELL DIAGNOSTICS, INC., Newark, CA (US)

(21) Appl. No.: 17/438,579

(22) PCT Filed: Mar. 11, 2020
(86) PCT No.: PCT/US20/22010

§ 371 Date: Sep. 13, 2021

Related U.S. Application Data
(60) Provisional application No. 62/817,449, filed on Mar. 12, 2019.

(51) Int. Cl. C12Q 1/6841; C12Q 1/6886; G01N 1/30

(52) U.S. Cl. CPC C12Q 1/6841; G01N 1/30; C12Q 1/6886

(57) ABSTRACT

The invention provides methods for detecting target double stranded nucleic acids, including for in situ hybridization assays. The invention also provides samples of fixed and permeabilized cells with a detected target double stranded nucleic acids as well as slides containing such samples. The invention additionally provides kits for detecting target double stranded nucleic acids.

### METHODS RELATED THERETO

This application claims the benefit of U.S. Provisional Application No. 62/817,449 filed Mar. 12, 2019, the entire contents of which are incorporated herein by reference.

### BACKGROUND OF THE INVENTION

The present invention relates generally to detection of nucleic acids, and more specifically to detection of double stranded nucleic acids in situ.

DNA in situ hybridization (ISH) is a molecular biology technique widely used for detecting a specific sequence in a chromosome, cell or tissue while preserving the chromosomal, cellular and tissue context (Ratan et al., Cureus 9(6):e1325. doi: 10.7759/cureus.1325 (2017)). It has numerous applications in research and diagnostics (Hu et al., Biomark. Res. 2(1):3. doi: 10.1186/2050-7771-2-3 (2014); Ratan et al., supra, 2017; Weier et al., Expert Rev. Mol. Diagn. 2(2):109-119 (2002)). However, current DNA ISH methods can only detect large chromosomal regions (>100 kb) due to their use of large probes and limited sensitivity for shorter sequences. Since the median gene size in the human genome is only 24 kilobases, this means that almost all current DNA ISH probes span more than one gene, which often makes it difficult to draw conclusions at the single gene level. Therefore, a more sensitive and specific method that allows for visualization of shorter sequences remains a technical challenge.

Thus, there exists a need for in situ detection methods for double stranded nucleic acids, particularly of smaller regions and or individual genes. The present invention satisfies this need and provides related advantages as well.

### SUMMARY OF INVENTION

The invention provides methods of detecting a double stranded nucleic acid. In one embodiment, the invention provides a method of detecting a double stranded nucleic acid comprising (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a pre-amplifier comprising a binding site for the first target probe, a binding site for the second target probe, and a plurality of binding sites for an amplifier; (C) contacting the sample with a plurality of amplifiers, wherein the amplifiers comprise a binding site for the pre-amplifier and a plurality of binding sites for a label probe; (D) contacting the sample with a plurality of label probes, wherein the label probe comprises a label and a binding site for the amplifier; and (E) detecting the label probes bound to the target double stranded nucleic acid, thereby detecting the target double stranded nucleic acid.

In one embodiment of such a method, the target probe set comprises two or more pairs of target probes that specifically hybridize to the same target nucleic acid.

In one embodiment of such a method of detecting a double stranded nucleic acid, the double stranded nucleic acid is DNA, RNA, or a DNA/RNA hybrid.

In one embodiment of such a method, the sample is a tissue specimen or is derived from a tissue specimen. In another embodiment of such a method, the sample is a blood sample or is derived from a blood sample. In yet another embodiment of such a method, the sample is a cytological sample or derived from a cytological sample.

In one embodiment, the inventions provides a sample of fixed and permeabilized cells, comprising (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-amplifier comprising a binding site for the first target probe, a binding site for the second target probe, and a plurality of binding sites for an amplifier, wherein the pre-amplifier is hybridized to the first and second target probes; (D) a plurality of amplifiers, wherein the amplifiers comprise a binding site for the pre-amplifier and a plurality of binding sites for a label probe, and wherein the amplifiers are hybridized to the pre-amplifier; and (E) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment, the sample is a blood sample or is derived from a blood sample. In yet another embodiment of such a sample, the sample is a cytological sample or derived from a cytological sample.

In one embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-amplifier comprising a binding site for the first target probe, a binding site for the second target probe, and a plurality of binding sites for an amplifier, wherein the pre-amplifier is hybridized to the first and second target probes; (D) a plurality of amplifiers, wherein the amplifiers comprise a binding site for the pre-amplifier and a plurality of binding sites for a label probe, and wherein the amplifiers are hybridized to the pre-amplifier; and (E) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In another embodiment of such a slide, the sample is a tissue specimen or is derived from a tissue specimen. In another embodiment, the sample is a blood sample or is derived from a blood sample. In yet another embodiment of such a slide, the sample is a cytological sample or derived from a cytological sample.

In one embodiment, the invention provides a kit for detection of a target double stranded nucleic acid, comprising (A) a pre-amplifier comprising a binding site for the first target probe, a binding site for the second target probe, and a plurality of binding sites for an amplifier; (B) a plurality of amplifiers, wherein the amplifiers comprise a binding site for the pre-amplifier and a plurality of binding sites for a label probe; and (C) a plurality of label probes, wherein the label probe comprises a label and a binding site for the amplifiers.

In one embodiment of such a kit, the kit comprises a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid. In one embodiment, the target probe set comprises two or more pairs of target probes that specifically hybridize to the same target nucleic acid.

In one embodiment, the kit comprises at least one reagent for permeabilizing cells.

In one embodiment, the invention provides a method of detecting a double stranded nucleic acid comprising (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier; (C) contacting the sample with a plurality of pre-amplifiers, wherein the pre-amplifiers comprise a binding site for the first and second pre-pre-amplifiers and a plurality of binding sites for an amplifier; (D) contacting the sample with a plurality of amplifiers, wherein the amplifiers comprise a binding site for the pre-amplifiers and a plurality of binding sites for a label probe; (E) contacting the sample with a plurality of label probes, wherein the label probe comprises a label and a binding site for the amplifier; and (F) detecting the label probes bound to the target double stranded nucleic acid, thereby detecting the target double stranded nucleic acid.

In one embodiment of such a method, the pre-amplifier comprises a binding site for the first and second pre-pre-amplifiers, wherein the melting temperature between the binding to both pre-pre-amplifiers is higher than the melting temperature between the binding of only one pre-pre-amplifier.

In one embodiment of such a method, the target probe set comprises two or more pairs of target probes that specifically hybridize to the same target nucleic acid.

In one embodiment of such a method, the double stranded nucleic acid is DNA, RNA, or a DNA/RNA hybrid.

In one embodiment of such a method, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a sample of fixed and permeabilized cells, comprising (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, wherein the first and second pre-pre-amplifiers comprise a plurality of binding sites for a pre-amplifier, and wherein the pre-pre-amplifiers are hybridized to the first and second target probes; (D) a plurality of pre-amplifiers, wherein the pre-amplifiers comprise a binding site for the first and second pre-pre-amplifiers and a plurality of binding sites for an amplifier, wherein the pre-amplifiers are hybridized to the first and send pre-pre-amplifiers; (E) a plurality of amplifiers, wherein the amplifiers comprise a binding site for the pre-amplifiers and a plurality of binding sites for a label probe, wherein the amplifiers are hybridized to the pre-amplifiers; and (F) a plurality of label probes, wherein the label probe comprises a label and a binding site for the amplifiers, wherein the label probes are hybridized to the amplifiers; wherein the hybridizations provide a detectable label on the target double stranded nucleic acid.

In one embodiment of such a sample, the pre-amplifier comprises a binding site for the first and second pre-pre-amplifiers, wherein the melting temperature between the binding to both pre-pre-amplifiers is higher than the melting temperature between the binding of only one pre-pre-amplifier.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier, and wherein the pre-pre-amplifiers are hybridized to the first and second target probes; (D) a plurality of pre-amplifiers, wherein the pre-amplifiers comprise a binding site for the first and second pre-pre-amplifiers and a plurality of binding sites for an amplifier, wherein the pre-amplifiers are hybridized to the first and second pre-pre-amplifiers; (E) a plurality of amplifiers, wherein the amplifiers comprise a binding site for the pre-amplifiers and a plurality of binding sites for a label probe, wherein the amplifiers are hybridized to the first and second pre-amplifiers; and (F) a plurality of label probes, wherein the label probe comprises a label and a binding site for the amplifiers, wherein the label probes are hybridized to the amplifiers; wherein the hybridizations provide a detectable label on the target double stranded nucleic acid.

In one embodiment of such a slide, the pre-amplifier comprises a binding site for the first and second pre-pre-amplifiers, wherein the melting temperature between the binding to both regions is higher than the melting temperature between the binding of only one region.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In one embodiment of such a slide, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a kit for detection of a target double stranded nucleic acid, comprising (A) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier; (B) a plurality of pre-amplifiers, wherein the pre-amplifiers comprise a binding site for the first and second pre-pre-amplifiers and a plurality of binding sites for an amplifier; (C) a plurality of amplifiers, wherein the amplifiers comprise a binding site for the pre-amplifiers and a plurality of binding sites for a label probe; and (D) a plurality of label probes, wherein the label probe comprises a label and a binding site for the amplifier.

In one embodiment of such a kit, the kit comprises a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid. In one embodiment, the target probe set comprises two or more pairs of target probes that specifically hybridize to the same target nucleic acid.

In one embodiment of such a kit, the pre-amplifier comprises a binding site for the first and second pre-pre-amplifiers, wherein the melting temperature between the binding to both regions is higher than the melting temperature between the binding of only one region.

In one embodiment, the kit comprises at least one reagent for permeabilizing cells.

In one embodiment, the invention provides a method of detecting a double stranded nucleic acid comprising: (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a pre-pre-amplifier comprising a binding site for the first target probe, a binding site for the second target probe, and a plurality of binding sites for a pre-amplifier; (C) contacting the sample with a plurality of pre-amplifiers comprising a binding site for the pre-pre-amplifier, and a plurality of binding sites for an amplifier; (D) contacting the sample with a plurality of amplifiers, wherein the amplifiers comprise a binding site for the pre-amplifier and a plurality of binding sites for a label probe; (E) contacting the sample with a plurality of label probes, wherein the label probe comprises a label and a binding site for the amplifier; and (F) detecting the label probes bound to the target double stranded nucleic acid, thereby detecting the target double stranded nucleic acid.

In one embodiment of such a method, the target probe set comprises two or more pairs of target probes that specifically hybridize to the same target nucleic acid.

In one embodiment of such a method, the double stranded nucleic acid is DNA, RNA, or a DNA/RNA hybrid.

In one embodiment of such a method, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a sample of fixed and permeabilized cells, comprising: (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-pre-amplifier comprising a binding site for the first target probe, a binding site for the second target probe, and a plurality of binding sites for a pre-amplifier, wherein the pre-pre-amplifier is hybridized to the first and second target probes; (D) a plurality of pre-amplifiers comprising a binding site for the pre-pre-amplifiers and a plurality of binding sites for an amplifier, wherein the pre-amplifiers are hybridized to the pre-pre-amplifier; (E) a plurality of amplifiers, wherein the amplifiers comprise a binding site for the pre-amplifiers and a plurality of binding sites for a label probe, and wherein the amplifiers are hybridized to the pre-amplifiers; and (F) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-pre-amplifier comprising a binding site for the first target probe, a binding site for the second target probe, and a plurality of binding sites for a pre-amplifier, wherein the pre-pre-amplifier is hybridized to the first and second target probes; (D) a plurality of pre-amplifiers comprising a binding site for the pre-pre-amplifier and a plurality of binding sites for an amplifier, wherein the pre-amplifier is hybridized to the pre-pre-amplifier; (E) a plurality of amplifiers, wherein the amplifiers comprise a binding site for the pre-amplifiers and a plurality of binding sites for a label probe, and wherein the amplifiers are hybridized to the pre-amplifiers; and (F) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In one embodiment of such a slide, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a kit for detection of a target double stranded nucleic acid, comprising (A) a pre-pre-amplifier comprising a binding site for the first target probe, a binding site for the second target probe, and a plurality of binding sites for a pre-amplifier; (B) a pre-amplifier comprising a binding site for the pre-pre-amplifier and a plurality of binding sites for an amplifier; (C) a plurality of amplifiers, wherein the amplifiers comprise a binding site for the pre-amplifier and a plurality of binding sites for a label probe; and (D) a plurality of label probes, wherein the label probe comprises a label and a binding site for the amplifiers.

In one embodiment of such a kit, the kit comprises a target probe set, wherein the target probe set comprises a pair of target probes comprising a first probe that specifically hybridizes to a first strand of a target double stranded nucleic acid, and a second probe that specifically hybridizes to the second strand of the target double stranded nucleic acid. In one embodiment, the target probe set comprises two or more pairs of target probes that specifically hybridize to the same target nucleic acid.

In one embodiment, the kit comprises at least one reagent for permeabilizing cells.

### BRIEF DESCRIPTION OF THE DRAWINGS

FIG. 1 shows a schematic of probe designs (double strand probes (ds-probes)) for detecting double stranded DNA, RNA or DNA/RNA hybrid. For each probe pair (depicted as “Z₁” and “Z₂”), the target-binding region of one probe binds to the sense strand whereas that of the other probe binds to the anti-sense strand of double stranded DNA, RNA or DNA-RNA hybrid. Simultaneous binding of each probe of the pair forms a binding site for the pre-amplifier (Panels A and B). Panels A and B show two alternative exemplary probe orientations. Panel C shows adaption of the same probe design for the BaseScope™ signal amplification system (Baker et al., Nat. Commun. 8(1):1998, doi: 10.1038/s41467-017-02295-5 (2017)). Panel C depicts binding of each member of the pair of target probes to the sense and antisense strands of the target nucleic acid, respectively. Each target probe binds to a separate pre-pre-amplifier (Panel C). Optionally, one or more probe pairs can be designed to target the same double stranded nucleic acid.

FIG. 2 shows a flow diagram for the steps for an exemplary in situ hybridization assay (left), and a modified assay using chemical denaturation (right) to denature double stranded nucleic acids. Briefly, the standard protocol starts with baking a slide at 60° C. followed by deparaffinization to remove the paraffin wax (“Bake+Dewax”). The slide is then treated with Epitope Retrieval buffer (“ER2”) at 88° C. to unmask the target(s), followed by protease digestion (Protease III). Then, target probes are hybridized to the slide (“Probe”), which is followed by sequential hybridization with pre-amplifiers, amplifiers and label probes (“AMPs”) and then detection of the target with the chromogen Fast Red (“Detection”). The modified protocol for DNA detection adds a chemical denaturation step (“Chemical DN”, 70% formamide in 2×SSC at 80° C. for 20 minutes).

FIG. 3 shows detection of fibroblast growth factor receptor 1 (FGFR1) genomic DNA detection requires both Z1 (sense strand binding) and Z2 (antisense strand binding) probes, which ensures specificity for double stranded DNA. DNA was detected as red punctate dots inside nuclei (blue).

FIG. 4 shows detection of different genes in cells (HEK293) and tissues (colon cancer, ovarian cancer, normal pancreas, normal breast) by ds-probes. DNA was detected as red punctate dots inside nuclei (blue).

FIGS. 5A-5C show a schematic of previously described methods of detecting a nucleic acid target using a signal generating complex (SGC). PPA, pre-pre-amplifier; PA, pre-amplifier; AMP, amplifier; LP, label probe.

### DETAILED DESCRIPTION OF THE INVENTION

Disclosed herein are methods for detecting double stranded nucleic acids, such as DNA, double stranded RNA and/or DNA/RNA hybrids. The methods provide for highly sensitive and specific detection of double stranded nucleic acids in a cell.

The recently developed RNA ISH technology called RNAscope™ uses specially designed oligonucleotide probes, sometimes referred to as “double-Z” or ZZ probes, in combination with a branched-DNA-like signal amplification system to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)). Such a probe design greatly improves the specificity of signal amplification because only when both probes in each pair bind to their intended target can signal amplification occur. However, a direct application of RNAscope™ for DNA detection is hampered by unwanted RNA detection because RNAscope™ probes cannot discriminate DNA from RNA targets. Although RNA can be eliminated by enzymatic (for example, RNase A) and chemical (for example, NaOH) methods, adding these steps can cause significant degradation of nuclear and cellular morphology and compromise DNA detection or detection of double stranded nucleic acids in an in situ detection assay.

Described herein is a probe design strategy that provides specific detection of double stranded DNA, RNA or DNA/RNA hybrids using the principles of RNAscope™ signal amplification system without interference from RNA. This is achieved by designing each probe pair to bind to both strands of double stranded nucleic acid, such as double stranded DNA, RNA or DNA/RNA hybrid. Single stranded RNA or DNA will not be detected because only one of the two probes in each pair will bind to it, which effectively prevents signal amplification and detection of single stranded RNA and single stranded DNA targets. The assay strategy allows highly sensitive and specific detection of very short DNA sequences (as short as 1 kb), providing a ˜100-fold higher resolution than current DNA FISH assays. Furthermore, the methods of the invention selectively detect double stranded DNA, which make it possible to distinguish double stranded DNA from single stranded DNA. The methods of the invention are also applicable to distinguishing double stranded RNA and/or RNA-DNA hybrid from single stranded DNA and/or RNA. In addition, since this method preserves RNA when detecting DNA, the methods can be used for simultaneous detection of RNA and DNA targets in the same sample of cells or tissue section.

The present invention relates to methods that allow for highly sensitive and specific detection of nucleic acid sequences in a cell, in particular double stranded nucleic acids. The methods of the invention have numerous practical applications (Hu et al., supra, 2014; Ratan et al., supra, 2017; Weier et al., supra, 2002). The methods of the invention can be used, for example, in physical mapping of DNA sequences in chromosomes; three dimensional (3D) mapping of spatial genome organization; detection of gene copy number gain (duplication and amplification), loss (deletion) and gene rearrangement (translocation and fusion) in diseased cells and tissues; prenatal, postnatal and pre-transplantation diagnosis of chromosomal abnormalities; cancer diagnosis and prognosis; companion diagnostics; and detection and identification of pathogens (for example, bacteria and viruses).

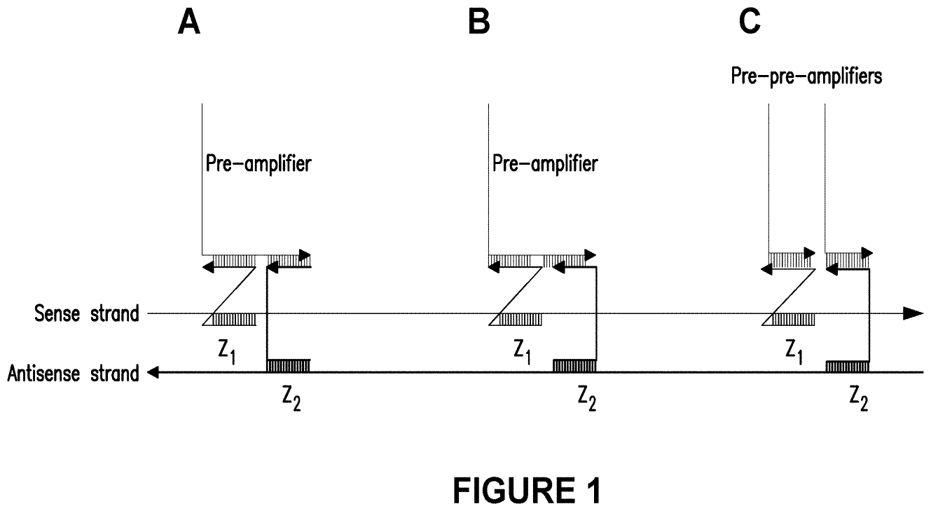

The present invention extends the probe design principle at the core of the RNAscope™ technology (Wang et al., supra, 2012) to double stranded nucleic acid, such as DNA, detection by requiring each probe pair to bind both strands of a double stranded nucleic acid, such as DNA, for signal amplification to occur (FIG. 1). Like RNAscope™ probes, each probe contains a sequence segment that binds to a specific sequence in the target. For double stranded nucleic acid detection, for example, DNA detection, two probes bind to adjacent sites on opposite strands in the target double stranded nucleic acid. Only when both probes bind to their respective target sites simultaneously can a full binding site for the signal amplification molecule (for example, a pre-amplifier as in FIGS. 1A and 1B or a pre-pre-amplifier as in FIG. 1C) be formed, leading to successful signal amplification and detection.

In FIG. 1, the target probe “Z₁” is depicted in a “Z” configuration, as described, for example, in U.S. Pat. No. 7,709,198, U.S. publications 2008/0038725 and 2009/0081688, and WO 2007/001986 and WO 2007/002006. The Z₁ configuration shown in FIG. 1 has the target binding site 5′ to the pre-amplifier or pre-pre-amplifier binding site of the target probe (FIGS. 1A-1C). It is understood that such a configuration, as depicted in FIG. 1, is merely exemplary, and the orientation can be the reverse, that is, the target binding site can be 3′ to the pre-amplifier or pre-pre-amplifier binding site. For example, as shown in FIG. 1A, probe “Z2” is depicted with the pre-amplifier binding site 5′ to the target binding site. In FIG. 1B, the probe “Z₂” depicts the target binding site 5′ to the pre-amplifier binding site. In FIG. 1C, the “Z₂” probe is similarly depicted with the target binding site 5′ to the pre-pre-amplifier binding site. It is understood that the target probe pair can independently be in either orientation, that is, one member of the pair of target probes can have the target binding site 5′ or 3′ to the pre-amplifier or pre-pre-amplifier binding site, and can be paired with a second probe having a binding site 5′ or 3′ to the pre-amplifier or pre-pre-amplifier, such that there are four possible combinations of orientations for the target probe pairs.

Also in FIG. 1, the two strands are labeled as “sense” and “antisense” for the purposes of illustration. It is understood that the target probe pairs bind to opposite strands of a double stranded nucleic acid. It is further understood that the region of a double stranded nucleic acid to be bound by the target probe pair does not need to be in a coding region, for example, a coding region of DNA having sense and antisense strands, but can be in a non-coding region.

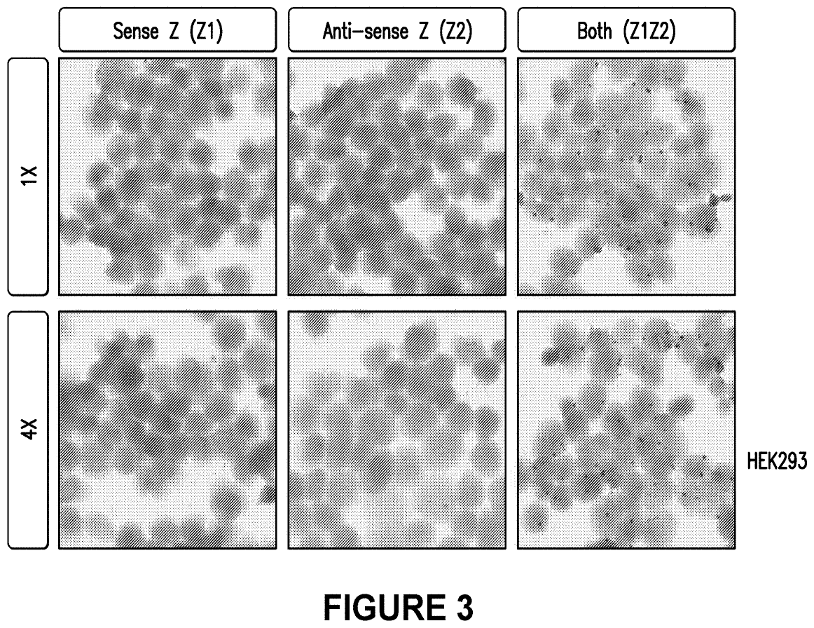

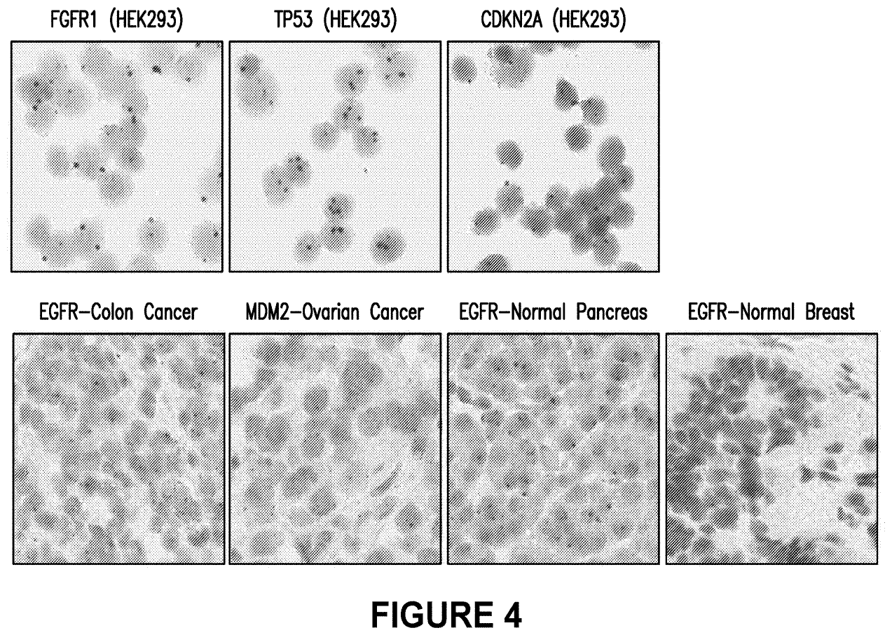

The effectiveness of the methods of the invention was demonstrated experimentally as described in Example 1 and shown in FIGS. 3 and 4. The same strategy can be adapted to use the BaseScope™ signal amplification system (Baker et al., supra, 2017) (FIG. 1C), further improving sensitivity and specificity. Other signal amplification methods such as hybridization chain reaction (HCR) (Choi et al., Development 145(12), pii: dev165753, doi: 10.1242/dev.165753 (2018)) can also be modified to incorporate the double stranded probe design principle as disclosed herein. The latest version of HCR (HCR v3.0) employs a similar paired probe design as RNAscope™ (Choi et al., supra, 2018). This technique can use similarly designed probe pairs binding to both DNA strands, as described herein, in order to initiate the hybridization chain reaction, which will ensure dsDNA specificity. Multiplexing to detect multiple double stranded nucleic acid targets, for example, multiple DNA targets, simultaneously on the same slide is straightforward with the use of either RNAscope™ or BaseScope™ technology. Thus, it is understood that various methods for detecting a nucleic acid in an in situ assay, such as those described herein and above, can be applied to the target probe configuration, in which a target probe pair binds to both strands of a target double stranded nucleic acid, as described herein for the detection of double stranded nucleic acids.

The methods of the invention disclosed herein can be used to detect any double stranded nucleic acid, such as DNA biomarkers, in various cells and tissue types. In one embodiment of the present invention, the ds-probe design is used with RNAscope™ to detect multiple gene targets in cells and tissues (see Example 1 and FIG. 4). The methods of the invention can be used to detect and characterize a variety of DNA structural variations such as deletion, insertion, gene fusion, translocation, duplication, and amplification found in neoplasms and other diseases. The methods of the invention can also be used to detect double stranded viral DNA and RNA and bacterial DNA with double-strand specificity.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, pre-amplifier, pre-pre-amplifier, signal generating complex (SGC), or the like, as described herein. In general in embodiments of the invention, the label probe binds to an amplifier. As used herein, a label probe comprising an enzyme label refers to a label probe comprising a nucleic acid binding portion such as an oligonucleotide and an enzyme that is coupled to the nucleic acid binding portion. As disclosed herein, the coupling of the enzyme to the nucleic acid binding portion can be covalent or through a high affinity binding interaction such as biotin/avidin or other similar high affinity binding molecules.

As used herein, a “target probe” is a polynucleotide that is capable of hybridizing to a target nucleic acid and capturing or binding a label probe or signal generating complex (SGC) component, for example, an amplifier, pre-amplifier or pre-pre-amplifier, to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. In general in embodiments of the invention, the target probe binds to a pre-amplifier, as in FIGS. 1A and 1B, or to a pre-pre-amplifier, as in FIG. 1C. The target probe is generally single stranded so that the complementary sequence is available to hybridize with a corresponding target nucleic acid, label probe, amplifier, pre-amplifier or pre-pre-amplifier. In embodiments of the invention, wherein the target nucleic is double stranded, the target probes are provided as a pair, wherein one member of the pair binds to one strand of the double stranded nucleic acid, and the other target probe binds to the opposite strand of the double stranded nucleic acid.

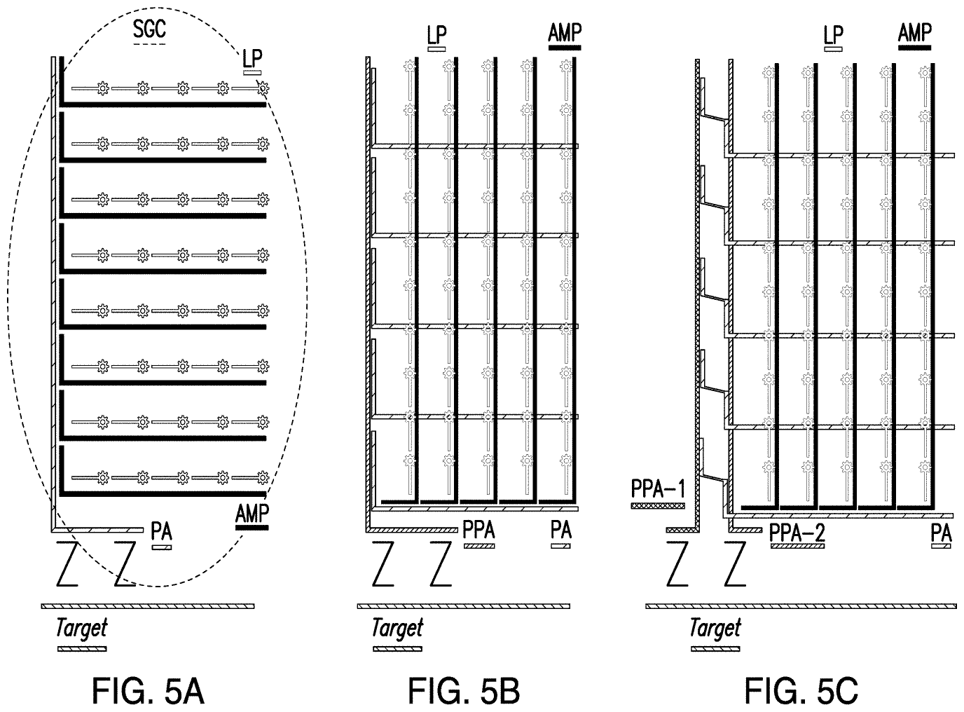

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. In general in embodiments of the invention, the amplifier can hybridize to a pre-amplifier. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference. In general in embodiments of the invention, the amplifier binds to a pre-amplifier and label probes (see FIG. 5).

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference. In general in embodiments of the invention, a pre-amplifier binds to both members of a target probe pair (see FIGS. 1A, 1B and 5A), to a pre-pre-amplifier that can bind to a target probe pair (FIG. 5B), or to both members of a pair of pre-pre-amplifiers that can bind to a target probe pair (see FIG. 5C). A pre-amplifier also binds to an amplifier (see FIG. 5).

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated by reference. In general in embodiments of the invention, a pre-pre-amplifier binds to a target probe pair (see FIG. 5B) or to a member of a target probe pair (see FIGS. 1C and 5C) and to a pre-amplifier (see FIG. 5C).

As used herein, the term “plurality” is understood to mean two or more. Thus, a plurality can refer to, for example, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 300 or more, 400 or more, 500 or more, 600 or more, 700 or more, 800 or more, 900 or more, or 1000 or more, or even a greater number, if desired for a particular use.

In one embodiment, the invention provides a method of detecting a double stranded nucleic acid comprising: (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a pre-amplifier comprising a binding site for the first target probe, a binding site for the second target probe, and a plurality of binding sites for an amplifier; (C) contacting the sample with a plurality of amplifiers, wherein the amplifiers comprise a binding site for the pre-amplifier and a plurality of binding sites for a label probe; (D) contacting the sample with a plurality of label probes, wherein the label probe comprises a label and a binding site for the amplifier; and (E) detecting the label probes bound to the target double stranded nucleic acid, thereby detecting the target double stranded nucleic acid.

In one embodiment, the target probe set comprises two or more pairs of target probes that specifically hybridize to the same target nucleic acid.

In one embodiment, the double stranded nucleic acid is DNA, RNA, or a DNA/RNA hybrid.

In one embodiment, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a method of detecting a double stranded nucleic acid comprising (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier; (C) contacting the sample with a plurality of pre-amplifiers, wherein the pre-amplifiers comprise a binding site for the first and second pre-pre-amplifiers and a plurality of binding sites for an amplifier; (D) contacting the sample with a plurality of amplifiers, wherein the amplifiers comprise a binding site for the pre-amplifiers and a plurality of binding sites for a label probe; (E) contacting the sample with a plurality of label probes, wherein the label probe comprises a label and a binding site for the amplifier; and (F) detecting the label probes bound to the target double stranded nucleic acid, thereby detecting the target double stranded nucleic acid.

In one embodiment, the pre-amplifier comprises a binding site for the first and second pre-pre-amplifiers, wherein the melting temperature between the binding to both pre-pre-amplifiers is higher than the melting temperature between the binding of only one pre-pre-amplifier.

the target probe set comprises two or more pairs of target probes that specifically hybridize to the same target nucleic acid.

In one embodiment, the double stranded nucleic acid is DNA, RNA, or a DNA/RNA hybrid.

In one embodiment, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a method of detecting a double stranded nucleic acid comprising (A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid; (B) contacting the sample with a pre-pre-amplifier comprising a binding site for the first target probe, a binding site for the second target probe, and a plurality of binding sites for a pre-amplifier; (C) contacting the sample with a plurality of pre-amplifiers comprising a binding site for the pre-pre-amplifier, and a plurality of binding sites for an amplifier; (D) contacting the sample with a plurality of amplifiers, wherein the amplifiers comprise a binding site for the pre-amplifier and a plurality of binding sites for a label probe; (E) contacting the sample with a plurality of label probes, wherein the label probe comprises a label and a binding site for the amplifier; and (F) detecting the label probes bound to the target double stranded nucleic acid, thereby detecting the target double stranded nucleic acid.

In one embodiment of such a method, the target probe set comprises two or more pairs of target probes that specifically hybridize to the same target nucleic acid.

In one embodiment of such a method, the double stranded nucleic acid is DNA, RNA, or a DNA/RNA hybrid.

In one embodiment of such a method, the sample is a tissue specimen or is derived from a tissue specimen. In another embodiment, the sample is a blood sample or is derived from a blood sample. In yet another embodiment, the sample is a cytological sample or is derived from a cytological sample.

In some embodiments of the invention, each target probe set that is specific for a target nucleic acid comprises two or more pairs of target probes that specifically hybridize to the same target double stranded nucleic acid. In such a case, the pairs of target probes in the target probe set specific for a target nucleic acid bind to different and non-overlapping sequences of the target nucleic acid. When a target probe set is used that has two or more pairs of target probes that can specifically hybridize to the same target nucleic acid, the molecule that binds to the target probe pairs, either a pre-amplifier (see FIGS. 1A, 1B and 5A), or a pre-pre-amplifier (see FIGS. 1C, 5B and 5C), generally are the same for target probe pairs in the same target probe set. Thus, the target probe pairs that bind to the same target double stranded nucleic acid can be designed to comprise the same binding site for the molecule in the SGC that binds to the target probe pairs, that is, a pre-amplifier or pre-pre-amplifier. The use of multiple target probe pairs to detect a target nucleic acid provides for a higher signal associated with the assembly of multiple SGCs on the same target nucleic acid. In some embodiments, the number of target probe pairs used for binding to the same target nucleic acid are in the range of 1-10, 1-20, 1-30, 1-40, 1-50, 1-60, 1-70, 1-80, 1-90, 1-100, 1-110, 1-120, 1-130, 1-140, 1-150, 1-160, 1-170, 1-180, 1-190, or 1-200 pairs per target, or larger numbers of pairs, or any integer number of pairs in between, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, and the like.

The methods of the invention can be utilized to achieve the detection of desired target double stranded nucleic acids. In one embodiment, a target nucleic acid is detected with a plurality of target probe pairs. In such a case, target probe pairs are designed to bind to more than one region of a target nucleic acid to allow for the assembly of multiple SGCs onto a target nucleic acid. It is understood that the target binding sites of one target probe pair do not overlap with the target binding sites of another target probe pair if a plurality of target probe pairs are being used to bind to the same target nucleic acid.

In an embodiment of the invention, the target nucleic acids detected by the methods of the invention can be any double stranded target nucleic acid present in the cell sample. In the methods of the invention for detection of target double stranded nucleic acids, it is understood the target nucleic acids can independently be DNA, double stranded RNA or DNA/RNA hybrids. Thus, the target nucleic acids to be detected can be, but are not necessarily, the same type of nucleic acid. In addition, the methods of the invention can be combined with methods for detecting single stranded nucleic acids, such as RNAscope™ or BaseScope™, such that both double stranded and single stranded nucleic acids can be detected in the same sample. The target nucleic acids include but not limited to, RNA, including messenger RNA (mRNA), micro RNA (miRNA), ribosomal RNA (rRNA), mitochondrial RNA, non-coding RNA, and the like, or DNA, and the like, or DNA/RNA hybrids. In the case where the target nucleic acids are RNA, it is understood that the target nucleic acids can independently be selected from the group consisting of messenger RNA (mRNA), micro RNA (miRNA), ribosomal RNA (rRNA), mitochondrial RNA, and non-coding RNA. Thus, the target nucleic acids can independently be DNA, either single stranded or double stranded, or any type of RNA, either signal stranded or double stranded, or DNA/RNA hybrids.

As described herein, the methods of the invention generally relate to in situ detection of double stranded nucleic acids. Methods for in situ detection of nucleic acids are well known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)).

For the methods of the invention for in situ detection of nucleic acid targets in a cell, including but not limited to in situ hybridization or flow cytometry, the cell is optionally fixed and/or permeabilized before hybridization of the target probes. Fixing and permeabilizing cells can facilitate retaining the nucleic acid targets in the cell and permit the target probes, label probes, amplifiers, pre-amplifiers, pre-pre-amplifiers, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, after hybridization of the pre-pre-amplifiers, pre-amplifiers, amplifiers, and/or label probes to the target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, Calif.), and enzymes (for example, lysozyme, lipases, proteases and peptidases). Permeabilization can also occur by mechanical disruption, such as in tissue slices.

For in situ detection of double stranded nucleic acids, generally the sample is treated to denature the double stranded nucleic acids in the sample to provide accessibility for the target probes to bind by hybridization to both strands of the target double stranded nucleic acid. Conditions for denaturing double stranded nucleic acids are well known in the art, and include heat and chemical denaturation, for example, with base (NaOH), formamide, dimethyl sulfoxide, and the like (see Wang et al., Environ. Health Toxicol. 29:e2014007 (doi: 10.5620/eht.2014.29.e2014007) 2014; Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999)). For example, NaOH, LiOH or KOH, or other high pH buffers (pH >11) can be used to denature double stranded nucleic acids such as DNA. In addition, heat and chemical denaturation methods can be used in combination.

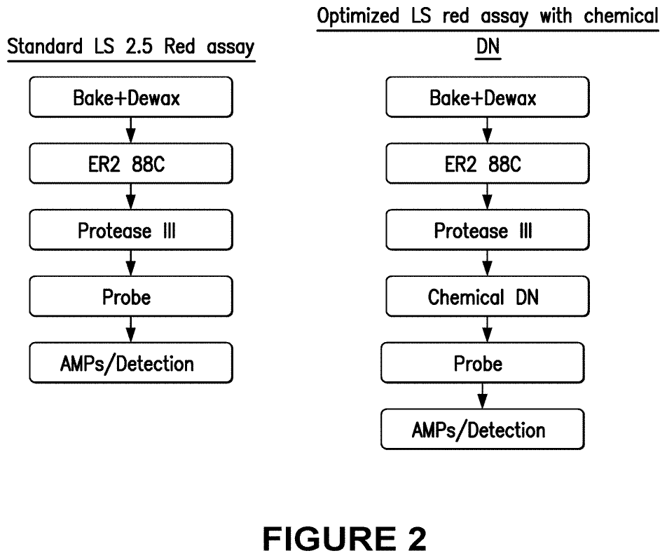

An exemplary assay protocol is outlined in FIG. 2. The assay depicted in FIG. 2 corresponds to the RNAScope™ 2.5 LS Red assay (user manual 322150, Advanced Cell Diagnostics, Newark Calif.) for use on a Leica Biosystems BOND RX system (Leica Biosystems, Buffalo Grove, Ill.). Briefly, the standard protocol starts with baking a slide at 60° C. and followed by deparaffinization to remove the paraffin wax (“Bake+Dewax”). The slide is then treated with Epitope Retrieval buffer (“ER2”) at 88° C. to unmask the target(s), followed by protease digestion (Protease III). Then, target probes are hybridized to the slide (“Probe”), which is followed by sequential hybridization with pre-amplifiers, amplifiers and label probes (“AMPs”) and then detection of the target with the chromogen Fast Red. The modified protocol for DNA detection adds a chemical denaturation step (“Chemical DN”, 70% formamide in 2×SSC at 80° C. for 20 minutes).

Such in situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the invention are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the invention can be used in diagnostic applications to identify the presence or absence of pathological cells based on the presence or absence of a double stranded nucleic acid target that is a biomarker indicative of a pathology.

It is understood by those skilled in the art that any of a number of suitable samples can be used for detecting target double stranded nucleic acids using methods of the invention. The sample for use in methods of the invention will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample, or cytological specimen. Such samples can be, but are not limited to, organs, tissues, tissue fractions and/or cells isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes. A sample can also be obtained from other sources, for example, food, soil, surfaces of objects, and the like, and other materials for which detection of double stranded nucleic acids is desired. Thus, the methods of the invention can be used for detection of one or more pathogens, such as a double stranded DNA or RNA virus, a bacterium, a fungus, a single celled organism, such as a parasite, and the like, from a biological sample obtained from an individual or other sources.

Collection of cytological samples for analysis by methods of the invention are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynocological Cytology Practice Guideline” American Society of Cytopathology, Adopted by the ASC executive board Mar. 2, 2004). Methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)). In one embodiment, the cytological sample is a cervical sample, for example, a pap smear. In one embodiment, the sample is a fine needle aspirate.

In particular embodiments of the invention, the sample is a tissue specimen or is derived from a tissue specimen. In other particular embodiments of the invention, the sample is a blood sample or is derived from a blood sample. In still other particular embodiments of the invention, the sample is a cytological sample or is derived from a cytological sample.

The invention is based on building a complex between a target double stranded nucleic acid in order to label the double stranded nucleic acid with a detectable label. Such a complex is sometimes referred to as a signal generating complex (SGC; see, for example, US 20170101672). Such a complex, or SGC, is achieved by building layers of molecules that allow the attachment of a large number of labels to a target double stranded nucleic acid.

The methods of the invention can employ a signal generating complex (SGC), where the SGC comprises multiple molecules rather than a single molecule. Such an SGC is particularly useful for amplifying the detectable signal, providing higher sensitivity detection of target nucleic acids. Such methods for amplifying a signal are described, for example, in U.S. Pat. Nos. 5,635,352, 5,124,246, 5,710,264, 5,849,481, and 7,709,198, and U.S. publications 2008/0038725 and 2009/0081688, as well as WO 2007/001986 and WO 2012/054795, each of which is incorporated herein by reference. The generation of an SGC is a principle of the RNAscope™ assay (see U.S. Pat. Nos. 7,709,198, 8,658,361 and 9,315,854, U.S. publications 2008/0038725, 2009/0081688 and 2016/0201117, as well as WO 2007/001986 and WO 2012/054795, each of which is incorporated herein by reference).

A basic Signal Generating Complex (SGC) is illustrated in FIG. 5A (see also US 2009/0081688, which is incorporated herein by reference). A pair of target probes, depicted in FIG. 5 as a pair of “Z's”, hybridizes to a complementary molecule sequence, labeled “Target”. For simplicity, FIG. 5 depicts the target as a single line, whereas it is understood that the target in the present invention is a double stranded nucleic acid, as illustrated in FIG. 1 in more detail. Each target probe contains an additional sequence complementary to a pre-amplifier molecule (PA, illustrated in green), which must hybridize simultaneously to both members of the target probe pair in order to bind stably. The pre-amplifier molecule is made up of two domains: one domain with a region that hybridizes to each target probe, and one domain that contains a series of nucleotide sequence repeats, each complementary to a sequence on the amplifier molecule (Amp, illustrated in black). The presence of multiple repeats of this sequence allows multiple amplifier molecules to hybridize to one pre-amplifier, which increases the overall signal amplification. Each amplifier molecule is made up of two domains, one domain with a region that hybridizes to the pre-amplifier, and one domain that contains a series of nucleotide sequence repeats, each complementary to a sequence on the label probe (LP, illustrated in yellow), allowing multiple label probes to hybridize to each amplifier molecule, further increasing the total signal amplification. Each label probe contains two components. One component is made up of a nucleotide sequence complementary to the repeat sequence on the amplifier molecule to allow the label probe to hybridize. This nucleotide sequence is linked to the second component, which can be any signal-generating entity, including a fluorescent or chromogenic label for direct visualization, a directly detectable metal isotope, or an enzyme or other chemical capable of facilitating a chemical reaction to generate a fluorescent, chromogenic, or other detectable signal, as described herein. In FIG. 5A, the label probe is depicted as a line, representing the nucleic acid component, and a star, representing the signal-generating component. Together, the assembly from target probe to label probe is referred to as a Signal Generating Complex (SGC).

FIG. 5B illustrates a SGC enlarged by adding an amplification molecule layer, in this case a pre-pre-amplifier molecule (PPA, shown in red). The PPA binds to both target probes in one domain and multiple pre-amplifiers (PAs) in another domain.

FIG. 5C illustrates a different SGC structure that uses collaborative hybridization at the pre-amplifier level (see US 2017/0101672, which is incorporated herein by reference). Similarly to the SGC formed in FIGS. 5A and 5B, a pair of target probes hybridize to the target molecule sequence. Each target probe contains an additional sequence complementary to a unique pre-pre-amplifier molecule (PPA-1, illustrated in purple; PPA-2, illustrated in red). The use of two independent molecules sets up a base on which collaborative hybridization can be required. Each pre-pre-amplifier molecule is made up of two domains, one domain with a region that hybridizes to one of the target probes, and one domain that contains a series of nucleotide sequence repeats, each containing both a sequence complementary to a sequence within the pre-amplifier molecule (PA, illustrated in green), as well as a spacer sequence to facilitate PPA-PA binding efficiency. To stably attach to the growing SGC, each PA must hybridize to both PPA molecules simultaneously. Each pre-amplifier molecule is made up of two domains, one domain that contains sequences complementary to both pre-pre-amplifiers to allow hybridization, and one domain that contains a series of nucleotide sequence repeats each complementary to a sequence on the amplifier molecule (AMP, illustrated in black). Multiple repeats of the amplifier hybridization sequence allows multiple amplifier molecules to hybridize to each pre-amplifier, further increasing signal amplification. For simplicity of illustration, amplifier molecules are shown hybridizing to one pre-amplifier molecule, but it is understood that amplifiers can bind to each pre-amplifier. Each amplifier molecule contains a series of nucleotide sequence repeats complementary to a sequence within the label probe (LP, illustrated in yellow), allowing several label probes to hybridize to each amplifier molecule. Each label probe contains a signal-generating element to provide for signal detection.

As described above, whether using a configuration as depicted in FIGS. 1A, 1B and 5A, or a configuration as depicted in FIGS. 1C and 5C, the components of the SGC are designed such that the binding of both target probes is required in order to build an SGC. In the case of the configuration of FIGS. 1A, 1B, 5A and 5B, a pre-amplifier (or pre-pre-amplifier in FIG. 5B) must bind to both members of the target probe pair for stable binding to occur. This is achieved by designing binding sites between the target probes and the pre-amplifier (or pre-pre-amplifier) such that binding of both target probes to the pre-amplifier (or pre-pre-amplifier) has a higher melting temperature (Tm) than the binding of a single target probe to the pre-amplifier (or pre-pre-amplifier), and where the binding of a single target probe is unstable under the conditions of the assay. This design has been described previously, for example, in U.S. Pat. No. 7,709,198, U.S. publications 2008/0038725 and 2009/0081688, WO 2007/001986 WO 2007/002006, Wang et al., supra, 2012, Anderson et al., supra, 2016). By configuring the SGC components this way, the assembly of the SGC is achieved when both target probes are bound to the target nucleic acid and the pre-amplifier, thereby reducing background noise since assembly of an SGC as a false positive is minimized.

In the case of the configuration of FIGS. 1C and 5C, the requirement that an SGC be formed only when both members of a target probe pair are bound to the target nucleic acid is achieved by requiring that a pre-amplifier be bound to both pre-pre-amplifiers, which in turn are bound to both members of the target probe pair, respectively. This requirement is achieved by designing the binding sites between the pre-pre-amplifiers and the pre-amplifier such that the melting temperature (Tm) between the binding of both pre-pre-amplifiers to the pre-amplifier is higher than the melting temperature of either pre-pre-amplifier alone, and where the binding of one of the pre-pre-amplifiers to the pre-amplifier is unstable under the conditions of the assay. This design has been described previously, for example, in US 20170101672, WO 2017/066211 and Baker et al., supra, 2017). Unless the pre-amplifier is bound to both pre-pre-amplifiers, the amplifiers and label probes cannot assemble into an SGC bound to the target nucleic acid, thereby reducing background noise since assembly of an SGC as a false positive is minimized.

As disclosed herein, the invention is based on building a signal-generating complex (SGC) bound to a target nucleic acid in order to detect the presence of the target nucleic acid in the cell. The components for building an SGC generally comprise nucleic acids such that nucleic acid hybridization reactions are used to bind the components of the SGC to the target nucleic acid. Methods of selecting appropriate regions and designing specific and selective reagents that bind to the target nucleic acids, in particular oligonucleotides or probes that specifically and selectively bind to a target nucleic acid, or other components of the SGC, are well known to those skilled in the art (see Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999)). The target probes are designed such that the probes specifically hybridize to a target nucleic acid. A desired specificity can be achieved using appropriate selection of regions of a target nucleic acid as well as appropriate lengths of a binding agent such as an oligonucleotide or probe, and such selection methods are well known to those skilled in the art. One skilled in the art will readily understand and can readily determine appropriate reagents, such as oligonucleotides or probes, that can be used to target one particular target nucleic acid over another target nucleic acid or over non-target nucleic acids, or to provide binding to the components of the SGC. Thus, it is understood that “specifically hybridize,” “specifically label,” and “specifically bind” (or grammatical variations thereof), refer to hybridization, labeling or binding to a target nucleic acid but not to non-target nucleic acids, for example, another target nucleic acid or nucleic acids that are not desired to be targeted. Similar specificity can be achieved for a target-specific SGC by using appropriate selection of unique sequences such that a given component of a target-specific SGC (for example, target probe, pre-pre-amplifier, pre-amplifier, amplifier, label probe) will bind to the respective components such that the target-specific SGC is bound to a specific target and not to another target nucleic acid.

As described herein, embodiments of the invention include the use of target probe pairs, with each member of the pair binding to opposite strands of a double stranded nucleic acid. In the case where a pair of target probes binds to the same pre-amplifier (FIGS. 1A, 1B and 5A), or pre-pre-amplifier (FIG. 5B), a probe configuration, sometimes referred to as a “Z” configuration, can be used. Such a configuration and its advantages for increasing sensitivity and decreasing background are described, for example, in U.S. Pat. No. 7,709,198, U.S. publications 2008/0038725 and 2009/0081688, and WO 2007/001986 and WO 2007/002006, each of which is incorporated herein by reference. U.S. Pat. No. 7,709,198 and U.S. publications 2008/0038725 and 2009/0081688 additionally describe details for selecting characteristics of the target probes, such as target probe pairs, including length, orientation, hybridization conditions, and the like. One skilled in the art can readily identify suitable configurations based on the teachings herein and, for example, in U.S. Pat. No. 7,709,198, U.S. publications 2008/0038725 and 2009/0081688, and WO 2007/001986 and WO 2007/002006.

As described herein, the target binding site of the target probes in a target probe pair can be in any desired orientation and combination. For example, the target binding site of one member of the target probe pair can be 5′ or 3′ to the pre-amplifier or pre-pre-amplifier binding site, and the other member of the pair can independently be oriented with the target binding site 5′ or 3′ to the pre-amplifier or pre-pre-amplifier binding site.

In another embodiment, the SGC used to detect the presence of a target double stranded nucleic acid is based on a collaborative hybridization of one or more components of the SGC (see US 20170101672 and WO 2017/066211, each of which is incorporated herein by reference). Such a collaborative hybridization is also referred to herein as BaseScope™. In a collaborative hybridization effect, the binding between two components of an SGC is mediated by two binding sites, and the melting temperature of the binding to the two sites simultaneously is higher than the melting temperature of the binding of one site alone (see US 20170101672 and WO 2017/066211). The collaborative hybridization effect can be enhanced by target probe set configurations as described in US 20170101672 and WO 2017/066211.

The methods of the invention, and related compositions, can utilize collaborative hybridization to increase specificity and to reduce background in in situ detection of double stranded nucleic acid targets, where a complex physiochemical environment and the presence of an overwhelming number of non-target molecules can generate high noise. Using such a collaborative hybridization method, the binding of label probes only occurs when the SGC is bound to the target nucleic acid. As described in US 20170101672 and WO 2017/066211 and illustrated in FIG. 1 thereof, the method can be readily modified to provide a desired signal to noise ratio by increasing the number of collaborative hybridizations in one or more components of the SGC.

In another embodiment, the collaborative hybridization can be applied to various components of the SGC. For example, the binding between components of an SGC can be a stable reaction, as described herein, or the binding can be configured to require a collaborative hybridization, also as described herein. In such a case, the binding component intended for collaborative hybridization are designed such that the component contains two segments that bind to another component.

Thus, the methods for detecting a target nucleic acid can utilize collaborative hybridization for the binding reactions between any one or all of the components in the detection system that provides an SGC specifically bound to a target nucleic acid. The number of components, and which components, to apply collaborative hybridization can be selected based on the desired assay conditions, the type of sample being assayed, a desired assay sensitivity, and so forth. Any one or combination of collaborative hybridization binding reactions can be used to increase the sensitivity and specificity of the assay. In embodiments of the invention, the collaborative hybridization can be between a pre-pre-amplifier and a pre-amplifier, between a pre-amplifier and an amplifier, between an amplifier and a label probe, or combinations thereof (see, for example, US 20170101672 and WO 2017/066211).

As disclosed herein, the components are generally bound directly to each other. In the case of nucleic acid containing components, the binding reaction is generally by hybridization. In the case of a hybridization reaction, the binding between the components is direct. If desired, an intermediary component can be included such that the binding of one component to another is indirect, for example, the intermediary component contains complementary binding sites to bridge two other components.

As described herein, the configuration of various components can be selected to provide a desired stable or collaborative hybridization binding reaction (see, for example, US 20170101672). It is understood that, even if a binding reaction is exemplified herein as a stable or unstable reaction, such as for a collaborative hybridization, any of the binding reactions can be modified, as desired, so long as the target double stranded nucleic acid is detected. It is further understood that the configuration can be varied and selected depending on the assay and hybridization conditions to be used. In general, if a binding reaction is desired to be stable, the segments of complementary nucleic acid sequence between the components is generally in the range of 10 to 50 nucleotides, or greater, for example, 16 to 30 nucleotides, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides, or greater. If a binding reaction is desired to be relatively unstable, such as when a collaborative hybridization binding reaction is employed, the segments of complementary nucleic acid sequence between the components is generally in the range of 5 to 18 nucleotides, for example, 5, 6, 7, 8, 9, 10, 11, 12, 13, 14, 15, 16, 17 or 18 nucleotides. It is understood that the nucleotide lengths can be somewhat shorter or longer for a stable or unstable hybridization, depending on the sequence (for example, GC content) and the conditions employed in the assay. It is further understood, as disclosed herein, that modified nucleotides such as Locked Nucleic Acid (LNA) or Bridged Nucleic Acid (BNA) can be used to increase the binding strength at the modified base, thereby allowing length of the binding segment to be reduced. Thus, it is understood that, with respect to the length of nucleic acid segments that are complementary to other nucleic acid segments, the lengths described herein can be reduced further, if desired. A person skilled in the art can readily determine appropriate probe designs, including length, the presence of modified nucleotides, and the like, to achieve a desired interaction between nucleic acid components.

In designing binding sites between two nucleic acid sequences comprising complementary sequences, the complementary sequences can optionally be designed to maximize the difference in melting temperature (dTₘ). This can be done by using melting temperature calculation algorithms known in the art (see, for example, SantaLucia, Proc. Natl. Acad. Sci. U.S.A. 95:1460-1465 (1998)). In addition, artificial modified bases such as Locked Nucleic Acid (LNA) or bridged nucleic acid (BNA) and naturally occurring 2′-O-methyl RNA are known to enhance the binding strength between complementary pairs (Petersen and Wengel, Trends Biotechnol. 21:74-81 (2003); Majlessi et al., Nucl. Acids Res. 26:2224-2229 (1998)). These modified bases can be strategically introduced into the binding site between components of an SGC, as desired.

One approach is to utilize modified nucleotides (LNA, BNA or 2′-O-methyl RNA). Because each modified base can increase the melting temperature, the length of binding regions between two nucleic acid sequences (i.e., complementary sequences) can be substantially shortened. The binding strength of a modified base to its complement is stronger, and the difference in melting temperatures (dTₘ) is increased. Yet another embodiment is to use three modified bases (for example, three LNA, BNA or 2′-O-methyl RNA bases, or a combination of two or three different modified bases) in the complementary sequences of a nucleic acid component or between two nucleic acid components, for example of a signal generating complex (SGC), that are to be hybridized. Such components can be, for example, pre-pre-amplifier, a pre-amplifier, an amplifier, a label probe, or a pair of target probes.

The modified bases, such as LNA or BNA, can be used in the segments of selected components of SGC, in particular those mediating binding between nucleic acid components, which increases the binding strength of the base to its complementary base, allowing a reduction in the length of the complementary segments (see, for example, Petersen and Wengel, Trends Biotechnol. 21:74-81 (2003); U.S. Pat. No. 7,399,845). Artificial bases that expand the natural 4-letter alphabet such as the Artificially Expanded Genetic Information System (AEGIS; Yang et al., Nucl. Acids Res. 34 (21): 6095-6101 (2006)) can be incorporated into the binding sites among the interacting components of the SGC. These artificial bases can increase the specificity of the interacting components, which in turn can allow lower stringency hybridization reactions to yield a higher signal.

With respect to a target probe pair, the target probe pair can be designed to bind to immediately adjacent segments of the target nucleic acid or on segments that have one to a number of bases between the target probe binding sites of the target probe pair. Generally, target probe pairs are designed for binding to the target nucleic acid such that there are generally between 0 to 500 bases between the binding sites on the target nucleic acid, for example, 0, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 420, 440, 460, 480, or 500 bases, or any integer length in between. In particular embodiments, the binding sites for the pair of target probes are between 0 and 100, for example, 0, 5, 10, 15, 20, 25, 30, 35, 40, 45, 50, 60, 70, 80, 90, 100, or any integer length in between. In the case of binding to a double stranded nucleic acid target, it is understood that the members of the target probe pair bind to opposite strands of the double stranded nucleic acid (see, for example, FIG. 1). Thus, it is understood that, when referring to immediately adjacent binding sites or binding sites that have a number of bases between the target probe binding sites of a double stranded target nucleic acid, the binding sites of the pair of target probes are on opposite strands. Unlike the binding of a pair of target probes to a single stranded target nucleic acid, in the case of a double stranded target it is understood that the target binding sites for the pair of probes can overlap since the binding sites occur on opposite strands for the respective members of the target probe pair. It is understood that overlap can occur so long as there is no steric hindrance for the simultaneous binding of both target probes to the respective target nucleic acid strands. A person skilled in the art can readily determine permissible overlap between target binding sites on opposite strands, if such overlap is desired.

The SGC also comprises a plurality of label probes (LPs). Each LP comprises a segment that is detectable. The detectable component can be directly attached to the LP, or the LP can hybridize to another nucleic acid that comprises the detectable component, i.e. the label. As used herein, a “label” is a moiety that facilitates detection of a molecule. Common labels in the context of the present invention include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment of the invention, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, Dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the invention, including detectable enzyme/substrate combinations (Pierce, Rockford Ill.; Santa Cruz Biotechnology, Dallas Tex.; Life Technologies, Carlsbad Calif.). In a particular embodiment of the invention, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford Ill.; Santa Cruz Biotechnology, Dallas Tex.; Invitrogen, Carlsbad Calif.; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord Calif.; biocare.net/products/detection/chromogens).

Exemplary rare earth metals and metal isotopes suitable as a detectable label include, but are not limited to, lanthanide (III) isotopes such as 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Dy, 162Dy, 163Dy, 164Dy, 165Ho, 166Er, 167Er, 168Er, 169Tm, 170Er, 171Yb, 172Yb, 173Yb, 174Yb, 175Lu, and 176Yb. Metal isotopes can be detected, for example, using time-of-flight mass spectrometry (TOF-MS) (for example, Fluidigm Helios and Hyperion systems, fluidigm.com/systems; South San Francisco, Calif.).

Biotin-avidin (or biotin-streptavidin) is a well known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway N.J.), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NETS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods of the invention can utilize concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the invention are well known in the art (see, for example, Johnson and Spence, “Molecular Probes Handbook, a Guide to Fluorescent Probes and Labeling Technologies, 11th ed., Life Technologies (2010)).

Well known methods such as microscopy, cytometry (e.g., mass cytometry, cytometry by time of flight (CyTOF), flow cytometry), or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

The methods of the invention can be utilized to achieve the detection of desired target double stranded nucleic acids. In one embodiment, a target nucleic acid is detected with a plurality of target probe pairs. In such a case, target probe pairs are designed to bind to more than one region of a target double stranded nucleic acid. It is understood that the target binding sites of one target probe pair do not overlap with the target binding sites of another target probe pair if a plurality of target probe pairs are being used to bind to the same target double stranded nucleic acid.

In another embodiment, the methods of the invention can be applied to multiplex detection of target double stranded nucleic acids. In one embodiment, the methods of the invention are applied to the detection of two or more target nucleic acids, for example, 2, 3, 4, 5, 6, 7, 8, 9 or 10 or more target nucleic acids. The number of target nucleic acids that can be detected depends on the detection label. For fluorescent labels, up to 10 nucleic targets can generally be detected. For metal tagged probes using mass spectrometry-based detection, the number of target nucleic acids can be up to 150. A person skilled in the art can readily select suitable distinct labels to allow detection of more than one target double stranded nucleic acid in a sample.

In still another embodiment, the methods of the invention can be applied to simultaneous detection of double stranded nucleic acids and single stranded nucleic acids, for example, detection of DNA and RNA in the same sample. In such a case, probes can be designed to detect single stranded nucleic acids, such as RNA (see, for example, U.S. Pat. No. 7,709,198, U.S. publications 2008/0038725 and 2009/0081688, and 2017/0101672) and double stranded nucleic acids, as described herein, such that both double stranded nucleic acids and single stranded nucleic acids, such as DNA and RNA, can be detected in the same sample.

The invention described herein generally relates to detection of double stranded nucleic acids in a sample. It is understood that the methods of the invention can additionally be applied to detecting target nucleic acids and optionally other molecules in the sample, in particular in the same cell as the target nucleic acid. For example, in addition to detecting target nucleic acids, proteins expressed in a cell can also concurrently be detected. Detection of proteins in a cell are well known to those skilled in the art, for example, by detecting the binding of protein-specific antibodies using any of the well known detection systems, including those described herein for detection of target nucleic acids. Detection of target nucleic acids and protein have been described (see, for example, Schulz et al., Cell Syst. 6(1):25-36 (2018)).

It is understood that the invention can be carried out in any desired order, so long as the double stranded target nucleic acid is detected. Thus, in a method of the invention, the steps of contacting a cell with any components for assembly of an SGC can be performed in any desired order, can be carried out sequentially, or can be carried out simultaneously, or some steps can be performed sequentially while others are performed simultaneously, as desired, so long as the target double stranded nucleic acid is detected. It is further understood that embodiments disclosed herein can be independently combined with other embodiments disclosed herein, as desired, in order to utilize various configurations, component sizes, assay conditions, assay sensitivity, and the like.

In some cases, it can be desirable to reduce the number of assay steps, for example, reduce the number of hybridization and wash steps. One way of reducing the number of assay steps is to pre-assemble some or all components of the SGC prior to contacting with a cell. Such a pre-assembly can be performed by hybridizing some or all of the components of the SGC together prior to contacting the target nucleic acid.

It is understood that the invention can be carried out in any format that provides for the detection of a target nucleic acid. Although implementation of the invention has generally been described herein using in situ hybridization, it is understood that the invention can be carried out for detection of target nucleic acids in other formats, in particular for detection of target nucleic acids in a cell, as are well known in the art. One method that can be used for detecting target nucleic acids in a cell is flow cytometry, as is well known in the art (see, for example, Shapiro, Practical Flow Cytometry 3rd ed., Wiley-Liss, New York (1995); Ormerod, Flow Cytometry, 2nd ed., Springer (1999)). The methods, samples and kits of the invention can thus be used in an in situ hybridization assay format or another format, such as flow cytometry. The application of nucleic acid detection methods, including in situ hybridization, to flow cytometry has been described previously (see, for example, Hanley et al., PLoS One, 8(2):e57002. doi: 10.1371/journal.pone.0057002 (2013); Baxter et al., Nature Protocols 12(10):2029-2049 (2017)).

In some cases, it can be desirable to reduce the number of assay steps, for example, reduce the number of hybridization and wash steps. One way of reducing the number of assay steps is to pre-assemble some or all components of the SGC prior to contacting with a cell. Such a pre-assembly can be performed by hybridizing some or all of the components of the SGC together prior to contacting the target nucleic acid.

The invention also provides a sample comprising a cell or a plurality of cells. The cell can optionally be fixed. The cells can optionally be permeabilized. Fixing and/or permeabilizing cells is particularly applicable to in situ hybridization assays.

The invention also provides in one embodiment a sample of fixed and permeabilized cells, comprising (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-amplifier comprising a binding site for the first target probe, a binding site for the second target probe, and a plurality of binding sites for an amplifier, wherein the pre-amplifier is hybridized to the first and second target probes; (D) a plurality of amplifiers, wherein the amplifiers comprise a binding site for the pre-amplifier and a plurality of binding sites for a label probe, and wherein the amplifiers are hybridized to the pre-amplifier; and (E) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment, the sample is a blood sample or is derived from a blood sample. In yet another embodiment, the sample is a cytological sample or is derived from a cytological sample.

In one embodiment, the invention provides a sample of fixed and permeabilized cells, comprising (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, wherein the first and second pre-pre-amplifiers comprise a plurality of binding sites for a pre-amplifier, and wherein the pre-pre-amplifiers are hybridized to the first and second target probes; (D) a plurality of pre-amplifiers, wherein the pre-amplifiers comprise a binding site for the first and second pre-pre-amplifiers and a plurality of binding sites for an amplifier, wherein the pre-amplifiers are hybridized to the first and send pre-pre-amplifiers; (E) a plurality of amplifiers, wherein the amplifiers comprise a binding site for the pre-amplifiers and a plurality of binding sites for a label probe, wherein the amplifiers are hybridized to the pre-amplifiers; and (F) a plurality of label probes, wherein the label probe comprises a label and a binding site for the amplifiers, wherein the label probes are hybridized to the amplifiers; wherein the hybridizations provide a detectable label on the target double stranded nucleic acid.

In one embodiment of such a sample, the pre-amplifier comprises a binding site for the first and second pre-pre-amplifiers, wherein the melting temperature between the binding to both pre-pre-amplifiers is higher than the melting temperature between the binding of only one pre-pre-amplifier.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment of such a sample, the sample is a blood sample or is derived from a blood sample. In yet another embodiment of such a sample, the sample is a cytological sample or is derived from a cytological sample.

In one embodiment, the invention provides a sample of fixed and permeabilized cells, comprising (A) at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-pre-amplifier comprising a binding site for the first target probe, a binding site for the second target probe, and a plurality of binding sites for a pre-amplifier, wherein the pre-pre-amplifier is hybridized to the first and second target probes; (D) a plurality of pre-amplifiers comprising a binding site for the pre-pre-amplifiers and a plurality of binding sites for an amplifier, wherein the pre-amplifiers are hybridized to the pre-pre-amplifier; (E) a plurality of amplifiers, wherein the amplifiers comprise a binding site for the pre-amplifiers and a plurality of binding sites for a label probe, and wherein the amplifiers are hybridized to the pre-amplifiers; and (F) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a sample, the target probe set comprises two or more pairs of target probes that specifically hybridize to the same target nucleic acid.

In one embodiment of such a sample, the double stranded nucleic acid is DNA, RNA, or a DNA/RNA hybrid.

In one embodiment of such a sample, the sample is a tissue specimen or is derived from a tissue specimen. In another embodiment, the sample is a blood sample or is derived from a blood sample. In yet another embodiment, the sample is a cytological sample or is derived from a cytological sample.

The invention additionally provides a slide comprising a cell or a plurality of cells. Optionally, the cell or cells are fixed to the slide. Optionally, the cell or cells are permeabilized. In particular embodiments, the cells on the slide are fixed and/or permeabilized for an in situ assay.

In another embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-amplifier comprising a binding site for the first target probe, a binding site for the second target probe, and a plurality of binding sites for an amplifier, wherein the pre-amplifier is hybridized to the first and second target probes; (D) a plurality of amplifiers, wherein the amplifiers comprise a binding site for the pre-amplifier and a plurality of binding sites for a label probe, and wherein the amplifiers are hybridized to the pre-amplifier; and (E) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In one embodiment of such a slide, the sample is a tissue specimen or is derived from a tissue specimen. In another embodiment of such a slide, the sample is a blood sample or is derived from a blood sample. In yet another embodiment of such a slide, the sample is a cytological sample or is derived from a cytological sample.

In one embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier, and wherein the pre-pre-amplifiers are hybridized to the first and second target probes; (D) a plurality of pre-amplifiers, wherein the pre-amplifiers comprise a binding site for the first and second pre-pre-amplifiers and a plurality of binding sites for an amplifier, wherein the pre-amplifiers are hybridized to the first and second pre-pre-amplifiers; (E) a plurality of amplifiers, wherein the amplifiers comprise a binding site for the pre-amplifiers and a plurality of binding sites for a label probe, wherein the amplifiers are hybridized to the first and second pre-amplifiers; and (F) a plurality of label probes, wherein the label probe comprises a label and a binding site for the amplifiers, wherein the label probes are hybridized to the amplifiers; wherein the hybridizations provide a detectable label on the target double stranded nucleic acid.

In one embodiment of such a slide, the pre-amplifier comprises a binding site for the first and second pre-pre-amplifiers, wherein the melting temperature between the binding to both pre-pre-amplifiers is higher than the melting temperature between the binding of only one pre-pre-amplifier.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In one embodiment of such a slide, the sample is a tissue specimen or is derived from a tissue specimen. In another embodiment of such a slide, the sample is a blood sample or is derived from a blood sample. In yet another embodiment of such a slide, the sample is a cytological sample or is derived from a cytological sample.

In one embodiment, the invention provides a slide comprising (A) a slide having immobilized thereon a plurality of fixed and permeabilized cells comprising at least one fixed and permeabilized cell containing a target double stranded nucleic acid; (B) a target probe set, wherein the target probe set comprises a pair of target probes comprising a first probe specifically hybridized to a first strand of a target double stranded nucleic acid, and a second probe specifically hybridized to the second strand of the double stranded nucleic acid; (C) a pre-pre-amplifier comprising a binding site for the first target probe, a binding site for the second target probe, and a plurality of binding sites for a pre-amplifier, wherein the pre-pre-amplifier is hybridized to the first and second target probes; (D) a plurality of pre-amplifiers comprising a binding site for the pre-pre-amplifier and a plurality of binding sites for an amplifier, wherein the pre-amplifier is hybridized to the pre-pre-amplifier; (E) a plurality of amplifiers, wherein the amplifiers comprise a binding site for the pre-amplifiers and a plurality of binding sites for a label probe, and wherein the amplifiers are hybridized to the pre-amplifiers; and (F) a plurality of label probes, wherein the label probe comprises a label and a binding site for the amplifier, wherein the label probes are hybridized to the amplifiers; wherein the hybridizations provide a detectable label on the target double stranded nucleic acid.

In one embodiment of such a slide, the target probe set comprises two or more pairs of target probes that specifically hybridize to the same target nucleic acid.

In one embodiment of such a slide, the double stranded nucleic acid is DNA, RNA, or a DNA/RNA hybrid.

In one embodiment of such a slide, the sample is a tissue specimen or is derived from a tissue specimen. In another embodiment, the sample is a blood sample or is derived from a blood sample. In yet another embodiment, the sample is a cytological sample or is derived from a cytological sample.

The invention also provides a kit comprising the components of an SGC, as described herein, where the kit does not include the target nucleic acid. Such a kit can comprise pre-amplifiers (PAs), amplifiers (AMPs) and label probes (LPs), and optionally pre-pre-amplifiers (PPAs), as disclosed herein. Optionally the kit can comprise target probes (TPs) directed to a particular target double stranded nucleic acid, or a plurality of target double stranded nucleic acids. The components of a kit of the invention can optionally be in a container, and optionally instructions for using the kit can be provided.

In one embodiment, the invention provides a kit for detection of a target double stranded nucleic acid, comprising (A) a pre-amplifier comprising a binding site for the first target probe, a binding site for the second target probe, and a plurality of binding sites for an amplifier; (B) a plurality of amplifiers, wherein the amplifiers comprise a binding site for the pre-amplifier and a plurality of binding sites for a label probe; and (C) a plurality of label probes, wherein the label probe comprises a label and a binding site for the amplifiers.

In one embodiment of such a kit, the kit comprises a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid. In one embodiment, the target probe set comprises two or more pairs of target probes that specifically hybridize to the same target nucleic acid.

In one embodiment, the kit comprises at least one reagent for permeabilizing cells.

In one embodiment, the invention provides a kit for detection of a target double stranded nucleic acid, comprising (A) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier; (B) a plurality of pre-amplifiers, wherein the pre-amplifiers comprise a binding site for the first and second pre-pre-amplifiers and a plurality of binding sites for an amplifier; (C) a plurality of amplifiers, wherein the amplifiers comprise a binding site for the pre-amplifiers and a plurality of binding sites for a label probe; and (D) a plurality of label probes, wherein the label probe comprises a label and a binding site for the amplifier.

In one embodiment of such a kit, the kit comprises a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid. In one embodiment, the target probe set comprises two or more pairs of target probes that specifically hybridize to the same target nucleic acid.

In one embodiment of such a kit, the pre-amplifier comprises a binding site for the first and second pre-pre-amplifiers, wherein the melting temperature between the binding to both pre-pre-amplifiers is higher than the melting temperature between the binding of only one pre-pre-amplifier.

In one embodiment of such a kit, the kit comprises at least one reagent for permeabilizing cells.

In one embodiment, the invention provides kit for detection of a target double stranded nucleic acid, comprising (A) a pre-pre-amplifier comprising a binding site for the first target probe, a binding site for the second target probe, and a plurality of binding sites for a pre-amplifier; (B) a pre-amplifier comprising a binding site for the pre-pre-amplifier and a plurality of binding sites for an amplifier; (C) a plurality of amplifiers, wherein the amplifiers comprise a binding site for the pre-amplifier and a plurality of binding sites for a label probe; and (D) a plurality of label probes, wherein the label probe comprises a label and a binding site for the amplifiers.

In one embodiment, the kit comprises a target probe set, wherein the target probe set comprises a pair of target probes comprising a first probe that specifically hybridizes to a first strand of a target double stranded nucleic acid, and a second probe that specifically hybridizes to the second strand of the target double stranded nucleic acid. In one embodiment, the target probe set comprises two or more pairs of target probes that specifically hybridize to the same target nucleic acid.

In one embodiment, the kit comprises at least one reagent for permeabilizing cells.

It is understood that modifications which do not substantially affect the activity of the various embodiments of this invention are also provided within the definition of the invention provided herein. Accordingly, the following examples are intended to illustrate but not limit the present invention.

### Example I

### In Situ Detection of Double Stranded Nucleic Acids

This example describes in situ detection of double stranded nucleic acids.

A pair of probes were designed for detection of the fibroblast growth factor receptor 1 (FGFR1) gene, essentially as shown in FIG. 1B. HEK293 cells were contacted with either the sense probe, the antisense probe or both and detected by in situ hybridization. An overview of the assay protocol is shown in FIG. 2 as optimized LS red assay with chemical denaturation (FIG. 2, right panel). The assay was performed essentially according to the manufacturer's assay conditions for RNAScope™ 2.5 LS red (Advanced Cell Diagnostics) for use with a Leica Biosystems BOND RX system (Advanced Cell Diagnostics manual 322150). For detection of double stranded nucleic acids, a denaturation step was included in 70% formamide in 2×SSC at 80° C. for 20 minutes (20×SSC—3M NaCl, 0.3 M sodium citrate, pH 7.0). As shown in FIG. 3, detection of FGFR1 genomic DNA requires both Z1 (sense strand binding) and Z2 (antisense strand binding) probes. The requirement of both sense and antisense strand binding probes provides specificity for double stranded DNA. DNA was detected as red punctate dots inside nuclei (blue).

In situ detection of additional exemplary genes was performed. HEK293 cells were contacted with sense and antisense probes to detect genomic DNA for FGFR1, tumor protein 53 (TP53, also referred to as p53), and cyclin dependent kinase inhibitor 2A (CDKN2A) and detected by in situ hybridization essentially as described above. As shown in FIG. 4, upper panel, FGFR1, TP53 and CDKN2A were detected as red punctate dots inside nuclei (blue).

Tissue samples were also tested for the ability to detect genes in situ. Tissue samples of colon cancer, normal pancreas and normal breast were contacted with sense and antisense probes for detection of epidermal growth factor receptor (EGFR) gene and detected by in situ hybridization essentially as described above. A tissue sample of ovarian cancer was contacted with sense and antisense probes for detection of E3 ubiquitin-protein ligase Mdm2 (MDM2) gene. As shown in FIG. 4, lower panel, various genes were detected in cell lines and various tissue samples. DNA was detected as red punctate dots inside nuclei (blue).

These results show that probe pairs for detection of double stranded nucleic acids can be used to detect genes in situ in cell lines and tissue samples.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method of detecting a double stranded nucleic acid comprising:
(A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid;
(B) contacting the sample with a pre-amplifier comprising a binding site for the first target probe, a binding site for the second target probe, and a plurality of binding sites for an amplifier;
(C) contacting the sample with a plurality of amplifiers, wherein the amplifiers comprise a binding site for the pre-amplifier and a plurality of binding sites for a label probe;
(D) contacting the sample with a plurality of label probes, wherein the label probe comprises a label and a binding site for the amplifier; and
(E) detecting the label probes bound to the target double stranded nucleic acid, thereby detecting the target double stranded nucleic acid.

2. The method of claim 1, wherein the target probe set comprises two or more pairs of target probes that specifically hybridize to the same target nucleic acid.

3. The method of claim 1 or 2, wherein the double stranded nucleic acid is DNA, RNA, or a DNA/RNA hybrid.

4. The method of any one of claims 1-3, wherein the sample is a tissue specimen or is derived from a tissue specimen.

5. The method of any one of claims 1-3, wherein the sample is a blood sample or is derived from a blood sample.

6. The method of any one of claims 1-3, wherein the sample is a cytological sample or is derived from a cytological sample.

7. A sample of fixed and permeabilized cells, comprising:
(A) at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a pre-amplifier comprising a binding site for the first target probe, a binding site for the second target probe, and a plurality of binding sites for an amplifier, wherein the pre-amplifier is hybridized to the first and second target probes;
(D) a plurality of amplifiers, wherein the amplifiers comprise a binding site for the pre-amplifier and a plurality of binding sites for a label probe, and wherein the amplifiers are hybridized to the pre-amplifier; and
(E) a plurality of label probes, wherein the label probe comprises a label and a binding site for the amplifier, wherein the label probes are hybridized to the amplifiers;
wherein the hybridizations provide a detectable label on the target double stranded nucleic acid.

8. The sample of claim 7, wherein the target probe set comprises two or more pairs of target probes that specifically hybridize to the same target nucleic acid.

9. The sample of claim 7 or 8, wherein the double stranded nucleic acid is DNA, RNA, or a DNA/RNA hybrid.

10. The sample of any one of claims 7-9, wherein the sample is a tissue specimen or is derived from a tissue specimen.

11. The sample of any one of claims 7-9, wherein the sample is a blood sample or is derived from a blood sample.

12. The sample of any one of claims 7-9, wherein the sample is a cytological sample or is derived from a cytological sample.

13. A slide comprising:
(A) a slide having immobilized thereon a plurality of fixed and permeabilized cells comprising at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a pre-amplifier comprising a binding site for the first target probe, a binding site for the second target probe, and a plurality of binding sites for an amplifier, wherein the pre-amplifier is hybridized to the first and second target probes;
(D) a plurality of amplifiers, wherein the amplifiers comprise a binding site for the pre-amplifier and a plurality of binding sites for a label probe, and wherein the amplifiers are hybridized to the pre-amplifier; and
(E) a plurality of label probes, wherein the label probe comprises a label and a binding site for the amplifier, wherein the label probes are hybridized to the amplifiers;
wherein the hybridizations provide a detectable label on the target double stranded nucleic acid.

14. The slide of claim 13, wherein the target probe set comprises two or more pairs of target probes that specifically hybridize to the same target nucleic acid.

15. The slide of claim 13 or 14, wherein the double stranded nucleic acid is DNA, RNA, or a DNA/RNA hybrid.

16. The slide of any one of claims 13-15, wherein the sample is a tissue specimen or is derived from a tissue specimen.

17. The slide of any one of claims 13-15, wherein the sample is a blood sample or is derived from a blood sample.

18. The slide of any one of claims 13-15, wherein the sample is a cytological sample or is derived from a cytological sample.

19. A kit for detection of a target double stranded nucleic acid, comprising:
(A) a pre-amplifier comprising a binding site for the first target probe, a binding site for the second target probe, and a plurality of binding sites for an amplifier;
(B) a plurality of amplifiers, wherein the amplifiers comprise a binding site for the pre-amplifier and a plurality of binding sites for a label probe; and
(C) a plurality of label probes, wherein the label probe comprises a label and a binding site for the amplifiers.

20. The kit of claim 19, wherein the kit comprises a target probe set, wherein the target probe set comprises a pair of target probes comprising a first probe that specifically hybridizes to a first strand of a target double stranded nucleic acid, and a second probe that specifically hybridizes to the second strand of the target double stranded nucleic acid.

21. The kit of claim 20, wherein the target probe set comprises two or more pairs of target probes that specifically hybridize to the same target nucleic acid.

22. The kit of any one of claims 19-21, wherein the kit comprises at least one reagent for permeabilizing cells.

23. A method of detecting a double stranded nucleic acid comprising:
(A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid;
(B) contacting the sample with a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier;
(C) contacting the sample with a plurality of pre-amplifiers, wherein the pre-amplifiers comprise a binding site for the first and second pre-pre-amplifiers and a plurality of binding sites for an amplifier;
(D) contacting the sample with a plurality of amplifiers, wherein the amplifiers comprise a binding site for the pre-amplifiers and a plurality of binding sites for a label probe;
(E) contacting the sample with a plurality of label probes, wherein the label probe comprises a label and a binding site for the amplifier; and
(F) detecting the label probes bound to the target double stranded nucleic acid, thereby detecting the target double stranded nucleic acid.

24. The method of claim 23, wherein the pre-amplifier comprises a binding site for the first and second pre-pre-amplifiers, wherein the melting temperature between the binding to both pre-pre-amplifiers is higher than the melting temperature between the binding of only one pre-pre-amplifier.

25. The method of claim 23 or 24, wherein the target probe set comprises two or more pairs of target probes that specifically hybridize to the same target nucleic acid.

26. The method of any one of claims 23-25, wherein the double stranded nucleic acid is DNA, RNA, or a DNA/RNA hybrid.

27. The method of any one of claims 23-26, wherein the sample is a tissue specimen or is derived from a tissue specimen.

28. The method of any one of claims 23-26, wherein the sample is a blood sample or is derived from a blood sample.

29. The method of any one of claims 23-26, wherein the sample is a cytological sample or derived from a cytological sample.

30. A sample of fixed and permeabilized cells, comprising:
(A) at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, wherein the first and second pre-pre-amplifiers comprise a plurality of binding sites for a pre-amplifier, and wherein the pre-pre-amplifiers are hybridized to the first and second target probes;
(D) a plurality of pre-amplifiers, wherein the pre-amplifiers comprise a binding site for the first and second pre-pre-amplifiers and a plurality of binding sites for an amplifier, wherein the pre-amplifiers are hybridized to the first and send pre-pre-amplifiers;
(E) a plurality of amplifiers, wherein the amplifiers comprise a binding site for the pre-amplifiers and a plurality of binding sites for a label probe, wherein the amplifiers are hybridized to the pre-amplifiers; and
(F) a plurality of label probes, wherein the label probe comprises a label and a binding site for the amplifiers, wherein the label probes are hybridized to the amplifiers;
wherein the hybridizations provide a detectable label on the target double stranded nucleic acid.

31. The sample of claim 30, wherein the pre-amplifier comprises a binding site for the first and second pre-pre-amplifiers, wherein the melting temperature between the binding to both pre-pre-amplifiers is higher than the melting temperature between the binding of only one pre-pre-amplifier.

32. The sample of claim 30 or 31, wherein the target probe set comprises two or more pairs of target probes that specifically hybridize to the same target nucleic acid.

33. The sample of any one of claims 30-32, wherein the double stranded nucleic acid is DNA, RNA, or a DNA/RNA hybrid.

34. The sample of any one of claims 30-33, wherein the sample is a tissue specimen or is derived from a tissue specimen.

35. The sample of any one of claims 30-33, wherein the sample is a blood sample or is derived from a blood sample.

36. The sample of any one of claims 30-33, wherein the sample is a cytological sample or is derived from a cytological sample.

37. A slide comprising:
(A) a slide having immobilized thereon a plurality of fixed and permeabilized cells comprising at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier, and wherein the pre-pre-amplifiers are hybridized to the first and second target probes;
(D) a plurality of pre-amplifiers, wherein the pre-amplifiers comprise a binding site for the first and second pre-pre-amplifiers and a plurality of binding sites for an amplifier, wherein the pre-amplifiers are hybridized to the first and second pre-pre-amplifiers;
(E) a plurality of amplifiers, wherein the amplifiers comprise a binding site for the pre-amplifiers and a plurality of binding sites for a label probe, wherein the amplifiers are hybridized to the first and second pre-amplifiers; and
(F) a plurality of label probes, wherein the label probe comprises a label and a binding site for the amplifiers, wherein the label probes are hybridized to the amplifiers;
wherein the hybridizations provide a detectable label on the target double stranded nucleic acid.

38. The slide of claim 37, wherein the pre-amplifier comprises a binding site for the first and second pre-pre-amplifiers, wherein the melting temperature between the binding to both pre-pre-amplifiers is higher than the melting temperature between the binding of only one pre-pre-amplifier.

39. The slide of claim 37 or 38, wherein the target probe set comprises two or more pairs of target probes that specifically hybridize to the same target nucleic acid.

40. The slide of any one of claims 37-39, wherein the double stranded nucleic acid is DNA, RNA, or a DNA/RNA hybrid.

41. The slide of any one of claims 37-40, wherein the sample is a tissue specimen or is derived from a tissue specimen.

42. The slide of any one of claims 37-40, wherein the sample is a blood sample or is derived from a blood sample.

43. The slide of any one of claims 37-40, wherein the sample is a cytological sample or is derived from a cytological sample.

44. A kit for detection of a target double stranded nucleic acid, comprising:
(A) a set of pre-pre-amplifiers comprising a first and second pre-pre-amplifier, wherein the first pre-pre-amplifier comprises a binding site for the first target probe, wherein the second pre-pre-amplifier comprises a binding site for the second target probe, and wherein the first and second pre-pre-amplifiers comprise a plurality of binding sites for a pre-amplifier;
(B) a plurality of pre-amplifiers, wherein the pre-amplifiers comprise a binding site for the first and second pre-pre-amplifiers and a plurality of binding sites for an amplifier;
(C) a plurality of amplifiers, wherein the amplifiers comprise a binding site for the pre-amplifiers and a plurality of binding sites for a label probe; and
(D) a plurality of label probes, wherein the label probe comprises a label and a binding site for the amplifier.

45. The kit of claim 44, wherein the kit comprises a target probe set, wherein the target probe set comprises a pair of target probes comprising a first probe that specifically hybridizes to a first strand of a target double stranded nucleic acid, and a second probe that specifically hybridizes to the second strand of the target double stranded nucleic acid.

46. The kit of claim 45, wherein the target probe set comprises two or more pairs of target probes that specifically hybridize to the same target nucleic acid.

47. The kit of any one of claims 44-46, wherein the pre-amplifier comprises a binding site for the first and second pre-pre-amplifiers, wherein the melting temperature between the binding to both pre-pre-amplifiers is higher than the melting temperature between the binding of only one pre-pre-amplifier.

48. The kit of any one of claims 44-47, wherein the kit comprises at least one reagent for permeabilizing cells.

49. A method of detecting a double stranded nucleic acid comprising:
(A) contacting a sample comprising a cell comprising one or more double stranded nucleic acids with a target probe set, wherein the target probe set comprises a pair of target probes comprising a first probe that specifically hybridizes to a first strand of a target double stranded nucleic acid, and a second probe that specifically hybridizes to the second strand of the double stranded nucleic acid;
(B) contacting the sample with a pre-pre-amplifier comprising a binding site for the first target probe, a binding site for the second target probe, and a plurality of binding sites for a pre-amplifier;
(C) contacting the sample with a plurality of pre-amplifiers comprising a binding site for the pre-pre-amplifier, and a plurality of binding sites for an amplifier;
(D) contacting the sample with a plurality of amplifiers, wherein the amplifiers comprise a binding site for the pre-amplifier and a plurality of binding sites for a label probe;
(E) contacting the sample with a plurality of label probes, wherein the label probe comprises a label and a binding site for the amplifier; and
(F) detecting the label probes bound to the target double stranded nucleic acid, thereby detecting the target double stranded nucleic acid.

50. The method of claim 49, wherein the target probe set comprises two or more pairs of target probes that specifically hybridize to the same target nucleic acid.

51. The method of claim 49 or 50, wherein the double stranded nucleic acid is DNA, RNA, or a DNA/RNA hybrid.

52. The method of any one of claims 49-51, wherein the sample is a tissue specimen or is derived from a tissue specimen.

53. The method of any one of claims 49-51, wherein the sample is a blood sample or is derived from a blood sample.

54. The method of any one of claims 49-51, wherein the sample is a cytological sample or is derived from a cytological sample.

55. A sample of fixed and permeabilized cells, comprising:
(A) at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a pre-pre-amplifier comprising a binding site for the first target probe, a binding site for the second target probe, and a plurality of binding sites for a pre-amplifier, wherein the pre-pre-amplifier is hybridized to the first and second target probes;
(D) a plurality of pre-amplifiers comprising a binding site for the pre-pre-amplifiers and a plurality of binding sites for an amplifier, wherein the pre-amplifiers are hybridized to the pre-pre-amplifier;
(E) a plurality of amplifiers, wherein the amplifiers comprise a binding site for the pre-amplifiers and a plurality of binding sites for a label probe, and wherein the amplifiers are hybridized to the pre-amplifiers; and
(F) a plurality of label probes, wherein the label probe comprises a label and a binding site for the amplifier, wherein the label probes are hybridized to the amplifiers;
wherein the hybridizations provide a detectable label on the target double stranded nucleic acid.

56. The sample of claim 55, wherein the target probe set comprises two or more pairs of target probes that specifically hybridize to the same target nucleic acid.

57. The sample of claim 55 or 56, wherein the double stranded nucleic acid is DNA, RNA, or a DNA/RNA hybrid.

58. The sample of any one of claims 55-57, wherein the sample is a tissue specimen or is derived from a tissue specimen.

59. The sample of any one of claims 55-57, wherein the sample is a blood sample or is derived from a blood sample.

60. The sample of any one of claims 55-57, wherein the sample is a cytological sample or is derived from a cytological sample.

61. A slide comprising:
(A) a slide having immobilized thereon a plurality of fixed and permeabilized cells comprising at least one fixed and permeabilized cell containing a target double stranded nucleic acid;
(B) a target probe set, wherein the target probe set comprises a pair of target probes comprising a first probe specifically hybridized to a first strand of a target double stranded nucleic acid, and a second probe specifically hybridized to the second strand of the double stranded nucleic acid;
(C) a pre-pre-amplifier comprising a binding site for the first target probe, a binding site for the second target probe, and a plurality of binding sites for a pre-amplifier, wherein the pre-pre-amplifier is hybridized to the first and second target probes;
(D) a plurality of pre-amplifiers comprising a binding site for the pre-pre-amplifier and a plurality of binding sites for an amplifier, wherein the pre-amplifier is hybridized to the pre-pre-amplifier;
(E) a plurality of amplifiers, wherein the amplifiers comprise a binding site for the pre-amplifiers and a plurality of binding sites for a label probe, and wherein the amplifiers are hybridized to the pre-amplifiers; and
(F) a plurality of label probes, wherein the label probe comprises a label and a binding site for the amplifier, wherein the label probes are hybridized to the amplifiers;
wherein the hybridizations provide a detectable label on the target double stranded nucleic acid.

62. The slide of claim 61, wherein the target probe set comprises two or more pairs of target probes that specifically hybridize to the same target nucleic acid.

63. The slide of claim 61 or 62, wherein the double stranded nucleic acid is DNA, RNA, or a DNA/RNA hybrid.

64. The slide of any one of claims 61-63, wherein the sample is a tissue specimen or is derived from a tissue specimen.

65. The slide of any one of claims 61-63, wherein the sample is a blood sample or is derived from a blood sample.

66. The slide of any one of claims 61-63, wherein the sample is a cytological sample or is derived from a cytological sample.

67. A kit for detection of a target double stranded nucleic acid, comprising:
(A) a pre-pre-amplifier comprising a binding site for the first target probe, a binding site for the second target probe, and a plurality of binding sites for a pre-amplifier;
(B) a pre-amplifier comprising a binding site for the pre-pre-amplifier and a plurality of binding sites for an amplifier;
(C) a plurality of amplifiers, wherein the amplifiers comprise a binding site for the pre-amplifier and a plurality of binding sites for a label probe; and
(D) a plurality of label probes, wherein the label probe comprises a label and a binding site for the amplifiers.

68. The kit of claim 67, wherein the kit comprises a target probe set, wherein the target probe set comprises a pair of target probes comprising a first probe that specifically hybridizes to a first strand of a target double stranded nucleic acid, and a second probe that specifically hybridizes to the second strand of the target double stranded nucleic acid.

69. The kit of claim 68, wherein the target probe set comprises two or more pairs of target probes that specifically hybridize to the same target nucleic acid.

70. The kit of any one of claims 67-69, wherein the kit comprises at least one reagent for permeabilizing cells.
